(19) United States
(12) Patent Application Publication
(10) Pub. No.: US 2022/0257343 A1
(43) Pub. Date: Aug. 18, 2022

(54) Extraoral Orthopaedic Device for the Direct Protraction of the Maxilla and the Indirect Protraction of the Mandible

(71) Applicant: Stylianos Koutzoglou, Rethymno (GR)

(72) Inventors: Stylianos Koutzoglou, Rethymno (GR); Eleni L. Koutzoglou, Rethymno (GR)

(73) Assignee: Stylianos Koutzoglou, Rethymno (GR)

(21) Appl. No.: 17/738,271

(22) Filed: May 6, 2022

Related U.S. Application Data
(63) Continuation of application No. PCT/GR2020/000050, filed on Oct. 7, 2020.

(30) Foreign Application Priority Data
Nov. 8, 2019 (GR) 20190100500

(51) Int. Cl. A61C 7/06; A61C 7/36

(52) U.S. Cl. CPC A61C 7/06; A61C 8/0096; A61C 7/36

(57) ABSTRACT

An extraoral orthopedic device for direct protraction of the maxilla and indirect protraction of the mandible of a patient, comprising a circular cranial support configured for placement around the periphery of the neurocranium of a patient encircling the forehead, two perpendicular girders adjustably coupled to the circular cranial support and extending in a downward direction on either side of the face of the patient, each of the two perpendicular girders having a plurality of openings extending diametrically therethrough and disposed distally relative to the circular cranial support; an intraoral mechanism disposed in the mouth of the patient; and a horizontal girder coupled through elastic tractions with the intraoral mechanism and adjustably supported within a respective slot of the plurality of openings in each of the two perpendicular girders.

### CROSS-REFERENCE TO RELATED APPLICATIONS

This patent application claims priority to International Patent Application No. PCT/GR2020/000050, filed on Oct. 7, 2020, and which claims priority to Greek Patent Application No. 20190100500, filed on Nov. 8, 2019, both of which are incorporated by reference herein in their entirety.

### BACKGROUND OF THE INVENTION

So far, the extraoral devices for the protraction of the maxilla commonly used, are the “masks” designed by Delaire (Delaire J. Confection du masque orthopedique. Rev Stomatol. 1971; 72(5):579-84.) (Delaire V J, Verdon P, Floor J. Ziele and Ergebnisse extraoraler Zuge in postero-anteriorer Richtung in Anwendung einer orthopadischen Maske bei der Behandlung von Fallen der Klasse III. Fortschr Kiefer Orthop 1976; 37:246-262.) and Pettit (Petit H. Adaptations following accelerated facial mask therapy in clinical alteration of the growth face. In: McNamara J A Jr, Ribbens K A, Howe R P (eds). Clinical Alteration of the Growing Face, monograph 14, Craniofacial Growth Series. Ann Arbor, Mich.: University of Michigan, 1983.), which are related only to the skeletal Class III anomaly. In both of these devices, the forehead and chin are used as support in order to apply elastic forces, which currently amount to approximately 400 gr per side.

This way, the protraction of the maxilla is achieved (action), while the simultaneous pressure (reaction) on the temporomandibular joint tissues (particularly on the condyle, articular disc and fossa) could lead to a TMJ derangement, as anterior displacement of the articular disc (development of TMJ sounds, pain, reduced mouth opening, etc.).

Extraoral orthopaedic devices of the same use (support on the frontal bone and the chin) are also the Turley (Turley P K. Orthopedic correction of Class III malocclusion with palatal expansion and custom protraction headgear. J Clin Orthod. 1988; 22(5):314-325.) “mask” and the Face Mask/Reverse-pull Headgear Tübinger Model (https://www.orthazone.com/Face-Mask-ReversePull-Headgear-T % C3% BCbinger-Model-dtu-109).

The “Sky Hook” headgear (https://www.google.com/search?client=firefox-b-d&q=sky+hook+head+gear) is also used for the protraction of the maxilla after its “rapid expansion”, which is supported on the cranial bones, parietal and occipital, as well as on the chin. The elastic forces applied to the maxilla originate mostly from its chin support.

Another known device is the Grummons face mask (Hegmann M, Rather A K. The Grummons face mask as an early treatment modality within a class III therapy concept. J Orofacial Orthop. 2003; 64(6):450-6.), which uses the forehead as support and, instead of the chin, the infraorbital zygomatic area. This type of support differentiates this device from all of the above-mentioned. The face mask produced by the Leone company (https://www.leone.it/english/services/download/Cat Orthodontic Eng.pdf) is of the same philosophy. While these devices are used with the aim to protract the maxilla after its rapid expansion, the therapy results are highly controversial, because of the use of the infraorbital zygomatic support areas. These areas of the face comprise the zygomaticomaxillary sutures, which are subjected to a reactive pressure, which generates the maxillary pulling action.

From time to time, devices have appeared for the maxillary protraction, which use as skeletal anchorage only the frontal bone of the skull. The “maxillary modified protraction headgear” (Alcan T, Keles A, Erverdi N. The effects of a modified protraction headgear on maxilla. Am J Orthod Dentofacial Orthop. 2000; 117:27-38) is one such example. The only advantage of this device is its support. The non-use of the mandible, as support for the protraction of the maxilla, eliminates the possible side-effect of TMJ derangement, common to devices of the type described above. The industrial production as well as the practical use of this device are extremely impeded by the personalized bending of the extraoral and intraoral wires separately in every patient, aiming, firstly to an easy insertion of the intraoral wires to the tubes of a concrete intraoral appliance and secondly to avoid the well-known side-effects during maxillary protraction.

In addition, nowadays, intraoral orthopaedic devices of skeletal support are widely used in the treatment of Angle Class III malocclusion. Mini-plates or titanium mini-plates are surgically placed in the cheekbone, between the canine and lateral incisor or underneath the lower incisors' apices. The skeletal anchorage in the maxilla could be two palatal mini-screws, which are incorporated in an appliance used for “rapid palatal expansion” and protraction of the maxilla, or could be titanium mini-plates placed on the infra-zygomatic crest above the buccal roots of the first permanent molar. Growth repression of the mandible occurs because of the simultaneous backward compression of the chin by these devices. The glenoid fossa is transformed with corresponding displacement of the condyle. These anatomic changes are not required in all Angle Class III malocclusion cases, such as in patients, who present a physiologic growth in their mandible in conjunction with maxillary retrognathism due to size or position of the maxilla. This is commonly the case in cleft lip and palate patients. In the cases, where these anatomic changes could be a positive outcome, their medical usefulness is lost, because of the compression of the TMJ tissues, which could lead to a TMJ dysfunction.

Extraoral orthopaedic devices supported on the frontal bone and the chin, in combination with titanium infra-zygomatic mini-plates have been used for the maxillary protraction. In all these three types of devices, the mandible is pushed backwards, while the maxilla is protracted. In addition to the risk of creating a TMJ derangement, as it is already mentioned above, in the majority of the cases present a Class III malocclusion, the mandible is placed in the face properly. Nevertheless, all these types of devices operate in a compensatory manner, pushing the mandible backwards, modifying its growth and influencing its function and the aesthetic of the face adversely. Functionally, besides the side-effects that can be developed by the compression of the TMJs, we should have in mind that the backward push of the mandible, followed by the tongue, could contribute to future problems of sleep apnea. Don't forget, that all orthodontic appliances, which are used in the therapy of sleep apnea, are designed to bring the mandible forward, with the tongue following this displacement of the mandible with a simultaneous relief of the airway in the oropharynx level.

### BRIEF SUMMARY OF THE INVENTION

The present disclosure describes an extraoral device for the direct protraction of the maxilla, by forward, downward or upward adjustments, as well as for the indirect protraction of the mandible.

Through this extraoral device, direct traction is applied to the maxilla, immediately after its “rapid expansion” using elastics which are attached to the extraoral device and a mechanism configured for such “rapid palatal expansion.” The mandible can also be protracted indirectly in patients with a skeletal Class II malposition of the jaws, when the extraoral device is combined with intraoral mechanisms (“rapid palatal expansion” and other mechanism in order to move the mandible forward, like: functional appliances, removable or fixed mechanism in the mandible in combination with Class II elastics or other mechanisms aiming to move the mandible forward etc.).

### BRIEF DESCRIPTION OF THE SEVERAL VIEWS OF THE DRAWING(S)

FIG. 1 is a front perspective illustration of an extraoral device in accordance with the disclosure.

FIG. 2 is a rear perspective illustration of the extraoral device of FIG. 1.

FIG. 3 is a top perspective illustration of the extraoral device of FIGS. 1 and 2.

FIG. 4 is a partial cutaway view of the extraoral device shown in FIG. 3.

FIGS. 5a and 5c illustrations of horizontal girders for an extraoral device in accordance with the disclosure.

FIG. 5b is an enlarged detail view of a mounting screw in accordance with the disclosure.

### DETAILED DESCRIPTION OF THE INVENTION

The present disclosure describes an extraoral device that provides improved stability over solutions proposed in the past, for example, the “orthopedic device for the protraction of the maxillary arc” described in WO2017089971, and the “maxillary protraction device” described in US2018028282. While these prior devices are correctly designed, because their use avoids TMJ compression, they are not as stable as they should be. The embodiments in accordance with the present disclosure provide greater stability in use due to the anatomical design of their cranial support, ergonomics, fast and more precise adjustment to the head of every patient, as well as in the direction of the elastic forces in order to protract the maxilla during its use.

The main structural elements of a device in accordance with the present disclosure are a carbon fiber plate and girders, which make the device lightweight, extremely durable and easy to use. Finally, the direct protraction of the maxilla, mainly in patients who present opisthognathic maxilla in the face (maxillary retrognathia), despite their Angle Class I or Class II dental relationship, is not mentioned in the above-mentioned prior art devices. In these malocclusions, the protracted maxilla can be held in its new position by the device of the present disclosure and after that, the mandible can be moved forward by using another intraoral mechanism. This is the only way, the aetiologic therapy of the maxillary retrognathia in Angle Class I and II malocclusions can be achieved.

Nowadays, in patients with Angle Class II malocclusion, many of whom are in a growth phase and their growth potential can be modified, the orthodontist uses such techniques to move the maxillary permanent molars distally, aiming to an Angle Class I molar relationship, ignoring many times the mandibular retrognathia. In the best cases, the orthodontist uses functional appliances, removable, fixed or hybrid types, to move the mandible forward. Even in these cases, the devices used are supported in the maxilla to reposition the mandible forward, which results in suppressing of the forward maxillary growth.

In Angle Class II malocclusion cases, in which the maxilla is located in a harmonic or in a retrognathic position within the face, which is apparent by the increased nasolabial angle in the profile of the patient, the same therapy techniques are used, aiming to an Angle Class I dental relationship, ignoring the skeletal data of the jaws, as parts of the face in its entirety. In order to create harmonic and juvenile relationships of the two jaw bones within the face, orthofacial surgical interventions are used in adults, moving mainly the maxilla and the mandible forward and secondarily creating an Angle Class I molar relationship.

In one aspect, the device in accordance with the present disclosure creates or provides a convenient extraoral orthopaedic mechanism, stable and precise in its adjustment and function, which in combination with an intraoral device is able to protract the maxilla directly, easily and without side-effects, as well as the mandible, indirectly, where it is necessary. It is contemplated that the device can be used in cooperative growing patients.

Nowadays, in an Angle Class II malocclusion patient it is virtually impossible for an orthodontist to firstly reposition the maxilla further forward and deteriorate temporarily the dental Class II relationship and afterwards to reposition the mandible much more forward, holding simultaneously the maxilla in its protracted position with our suggested extraoral device.

Indeed, there is a plethora of Angle Class II malocclusion growing patients, with both jaw bones in a retrognathic position who can have their facial growth modified. Nevertheless, the orthodontist “going with the flow” holds the maxilla in its original retrognathic location within the face and with its support tries to relocate the mandible forward.

The proper head and body posture, the unimpeded nasal breathing, the mastication of not only soft food and the physiologic swallow pattern should be very seriously considered, in the context of a holistic orthodontic treatment.

In cases, where the original location of the maxilla within the face is in a prognathic position (severe labial inclination of the upper permanent incisors, very reduced nasolabial angle) or in the true skeletal Class III cases, in which the mandible is prognathic because of its size with respect to the cranial base, use of popular devices, with all their shortcomings, is accepted.

In contrast to existing devices, the extraoral orthopaedic device in accordance with the disclosure can be especially used, in cooperative growing patients, for whom the modification of the growth of their stomatognathic system is possible, for:

a. The therapy of skeletal Class III malocclusion, protracting the maxilla directly after its “rapid expansion” with the alternate way without the simultaneous compression and impeding of the mandible's growth, avoiding so: probable dysfunction of the TMJs, reducing of the tongue's vital space, which could lead in turn to a possible sleep apnea etc.

b. The therapy of skeletal Class II malocclusion, with Angle Class I or II dental relationship, in the cases the maxilla is retrognathic in its original position within the face. After the activation of the viscerocranium's sutures using the “rapid palatal expansion” technique with the alternate way, the maxilla is protracted initially and held in its relocated position subsequently by our extraoral device. Finally, the mandible is protracted using one more intraoral mechanism. In this way, the growth modification of both, maxilla and mandible, gives an unmatched aesthetic result in the appearance of the whole face and greatly helps the function of breathing and tongue, after the space in the mouth cavity has significantly increased. So, the probable cause of sleep apnea is notably reduced.

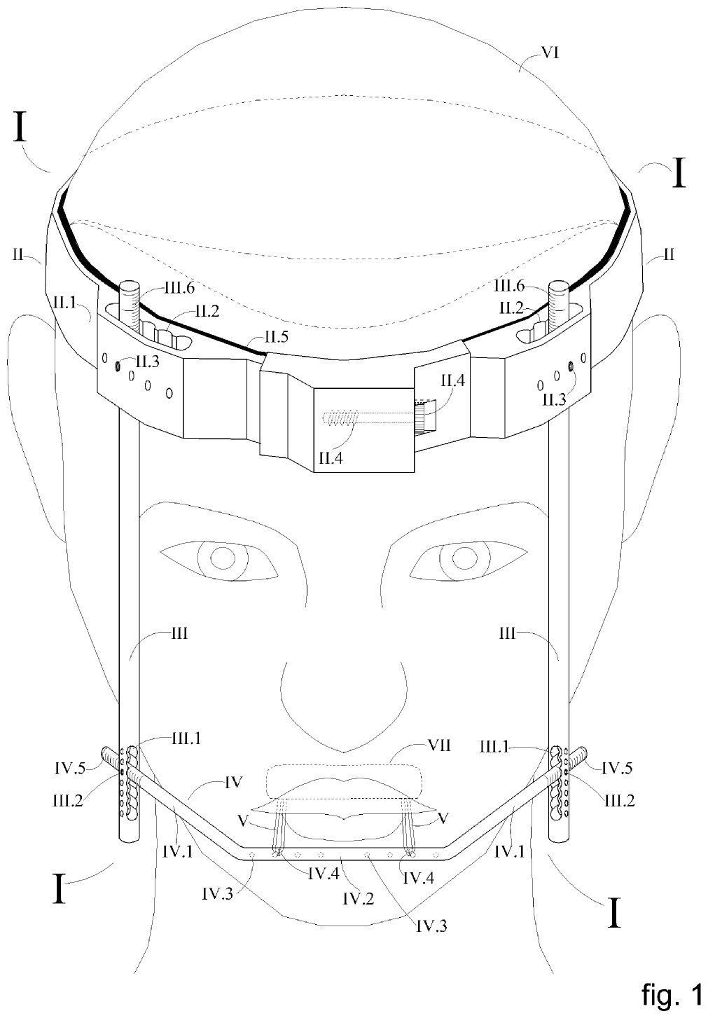

The device (I) in accordance with the disclosure, as illustrated in FIGS. 1-5, includes essentially 4 parts or structures, which are: the circular cranial support (II) placed exclusively on the periphery of the neurocranium, the perpendicular girders (III), the horizontal girder (IV) and the elastic tractions (V), through which the device (I) is coupled with the intraoral mechanism (VII) of “rapid palatal expansion”.

In FIG. 1 schematically illustrated are: the front view of the device (I) in use, the circular cranial support (II), the cylindrical girders (III) perpendicular to the circular cranial support (II), the horizontal, bent in two points, cylindrical girder and semi-cylindrical in its middle part between the bent points (IV). The horizontal girder (IV) is coupled with the circular cranial support (II) through the perpendicular cylindrical girders (III). In its semi-cylindrical part there are the supporting threaded holes (IV.3) and the mounting screws (IV.4), which are secured in the supporting holes and used for the attachment of the elastic tractions (V). The head of the patient (VI) and the intraoral mechanism of “rapid palatal expansion” (VII) are also illustrated. In the circular cranial support (II) on the head of the patient illustrated are: its tightening screw (II.4), the slots (II.2) to adjust the perpendicular girders (III) ergonomically and symmetrically and the fixing screws (II.3) in order to immobilize the perpendicular cylindrical girders, after their final adjustment. The markings (III.6), responsible for the symmetrical adjustment in height, of the right and left perpendicular girder (III) are also illustrated. Finally, illustrated are the slots (III.1) in the perpendicular girders, where the fast, precise and symmetric in height placement of the horizontal girder (IV) is attained, the fixing screws (III.2) for the immobilization of the horizontal girder after its final adjustment and the markings (IV.5) in the horizontal girder for its symmetric placement within the vertical girders (III) in the sagittal plane on the right and left side of the patient's head.

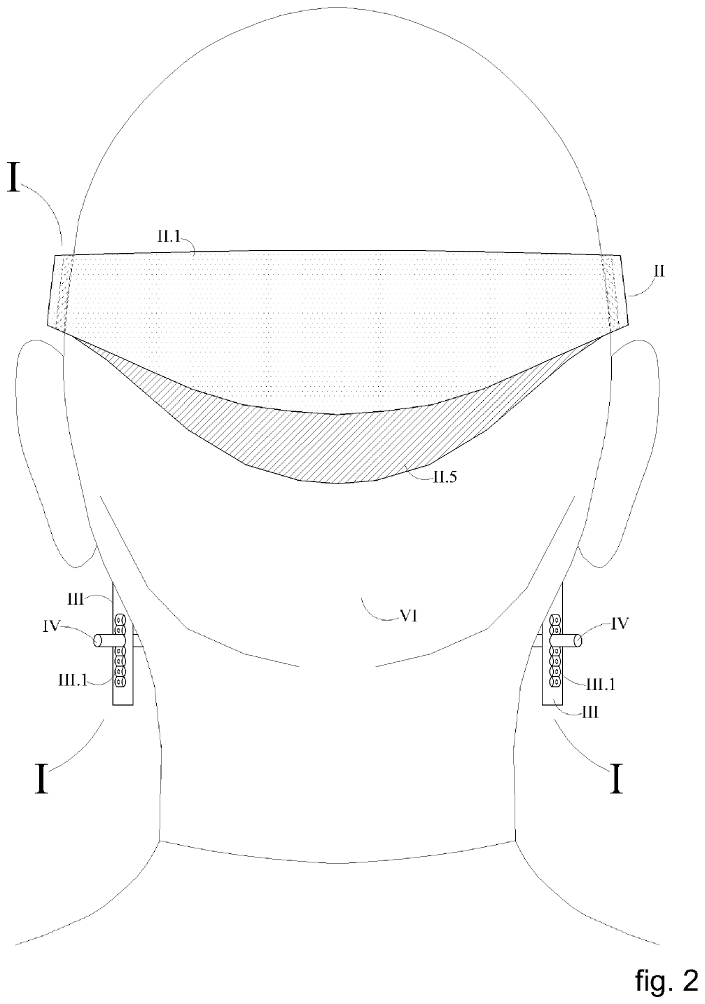

In FIG. 2, the rear view of the device (I) is shown, and also the patient's head (VI), the dorsal aspect of the circular cranial support (II) with its back very thin and tough segment (II.1—carbon fiber plate) and internal soft segment (II.5—medical silicon), as well as the region and the way of coupling of the two perpendicular cylindrical girders (III) with the horizontal girder (IV).

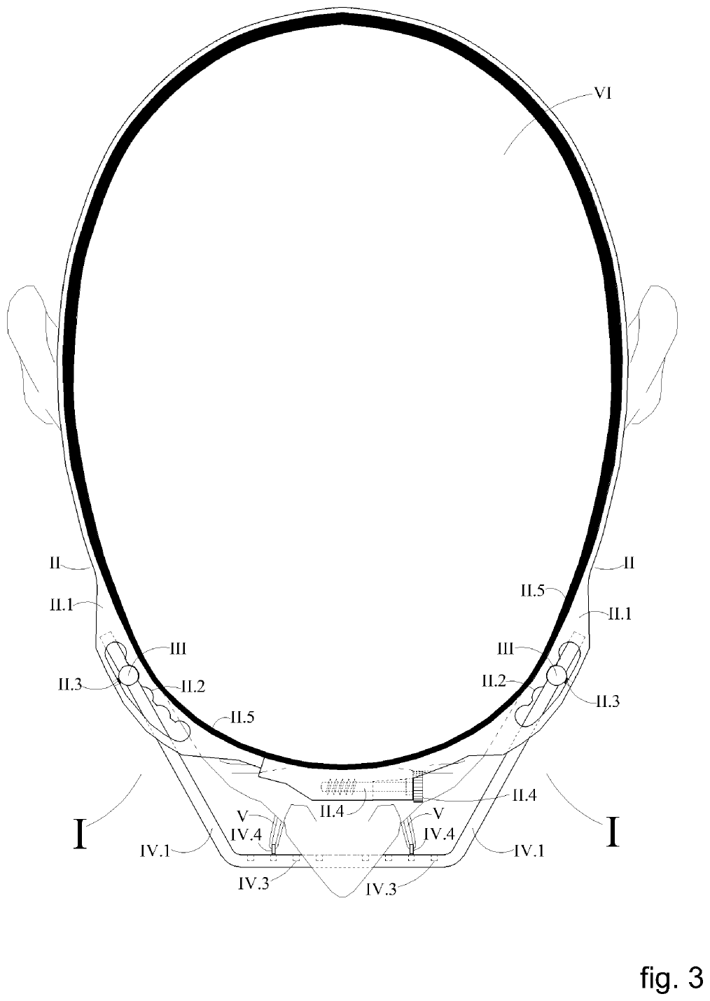

In FIG. 3, the upper aspect of our device (I) with its circular cranial support (II) in the frontal bone area and in the periphery of the skull is illustrated. Its external tough carbon fiber segment (II.1), its internal soft medical silicon inlay (II.5), the vertical positions-slots (II.2) for the placement of the perpendicular girders, the fixing screws (II.3) for the immobilization of the perpendicular girders after their individual adjustment to the patient's head, the fixing screw (II.4) of the circular cranial support (II) for the skeletal anchorage of our device corresponding to the individualized periphery of the patient's head and the upper view of the horizontal girder (IV) with its sockets in the form of internal threads (IV.3) and the mounting screws (IV.4), where the elastic bands (V) attach out of the patient's mouth, are also illustrated.

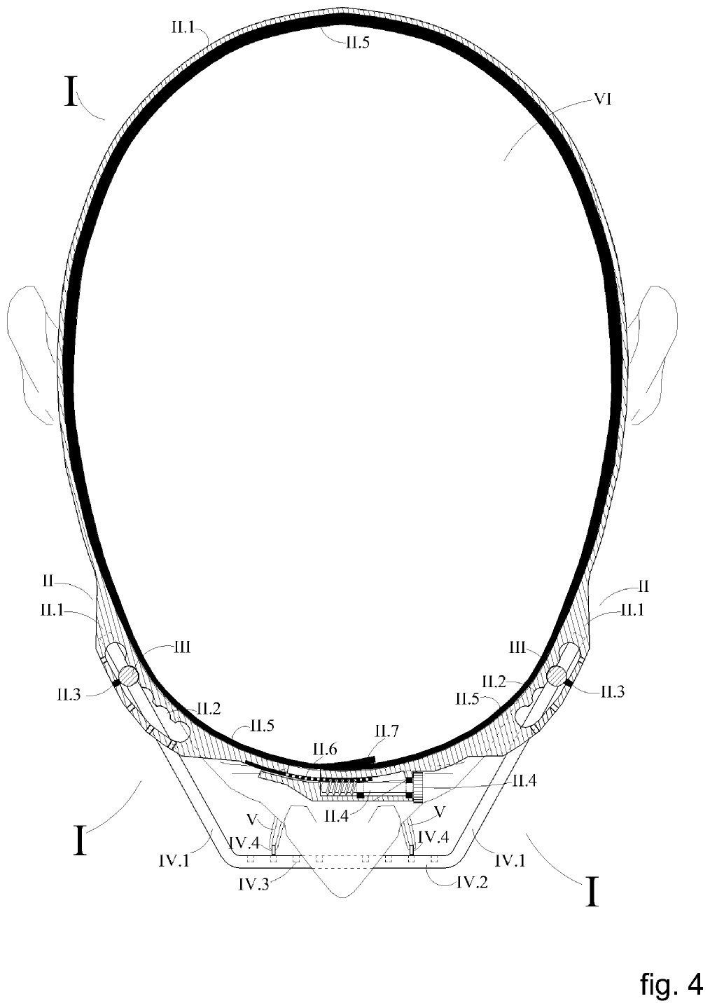

In FIG. 4, the transverse section of our device (I) in the height of the circular cranial support (II) in the frontal bone area and peripherally of the skull is illustrated. The front region of the patient's head is specifically illustrated. In this view, the clamping mechanism (II.6) [25] responsible for the adequate support of the circular cranial support (II) through its fixing screw (II.4), as well as the soft medical silicon portion of excess (II.7), which could be cut out by the orthodontist because of circular cranial support's tightening, after the final adjustment of the orthopaedic device (I) to the patient's head, are precisely shown.

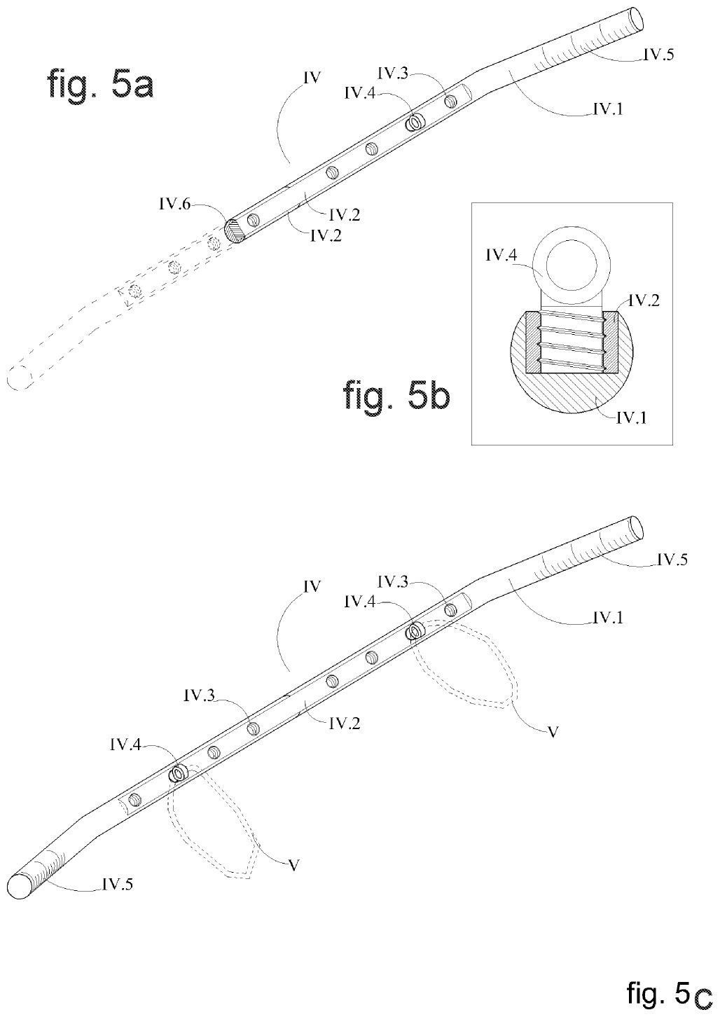

In FIG. 5, the horizontal girder (IV) with its construction details is illustrated: its carbon fiber segment (IV.1), the integrated metallic bar (IV.2) within the carbon fiber segment, which accommodates internal threads (IV.3) for the mounting screws (IV.4), by which the elastic tractions (V) are extraorally attached. The markings (IV.5) in the two ends of the horizontal girder (IV) aim to achieve direct, easy and absolute symmetry on the right and left side of the patient's head by adjusting the horizontal girder in the sagittal plane. Finally, the vertical section (IV.6) of the horizontal girder in its semi-cylindrical area, in which the metallic bar (IV.2) is integrated.

Initially, “rapid palatal expansion” with the alternate way is performed by any intraoral device. As an example, we mention the “Hyrax” device banded to the maxillary posterior teeth by glass-ionomer cement. In the buccal aspects of the bands metallic bars are welded, which have a circular bend at their front ends, in the canine area or a bit behind it, in order to be intraorally attached to the elastic tractions. On the 1st permanent maxillary molars and on the 2nd deciduous maxillary molars, fixed bite-planes of a visible light-cure material about 3 mm in height are placed to impede the maxillary molar extrusion, which is followed by downward and backward rotation of the mandible, by opening of the mid-palatal suture by the use of the “Hyrax” device. The bite planes are controlled in every visit for the case of occlusal imprints of the mandibular teeth, which obstruct the forward maxillary movement and they are repaired accordingly.

The extraoral orthopaedic device (I, FIG. 1-4) is applied immediately after the disarticulation of the circumaxillary sutures aiming to an adequate maxillary protraction.

At first, the circular cranial support (II, FIG. 1-4), which consists of a carbon fiber plate of different width and thickness is placed on the skull (VI, FIG. 1-4). After fixing it, to the periphery of the patient's head, through the clamping mechanism (II.4, II.6, FIG. 1, 3, 4), follows the adjustment of the remaining parts of our device.

The front part of the circular cranial support (II, FIG. 1), on the frontal bone and laterally distal of the lateral canthus, is more massive in order to accommodate the clamping mechanism (II.4, FIGS. 1, 3, 4 and II.6, FIG. 4) in its central part and laterally the perpendicular cylindrical girders (III, FIG. 1) through vertical slots (II.2, FIG. 1) for fastening in distinct positions. In every slot (II.2) corresponds a fixing screw (II.3).

Firstly, the perpendicular cylindrical girders are placed in their proper positions according to the patient's face width symmetrically to the right and left side of the patient's head. Afterwards, they are adjusted in height on the right and left side perfectly symmetrically, by means of the markings (111.6, FIG. 1) they have in their top ends and are finally immobilized by small fixing screws (11.3, FIG. 1).

The part of the circular cranial support (II.1, FIG. 1) right behind the slots, (II.2, FIG. 1, 3, 4) responsible for the proper adjustment and immobilization of the perpendicular cylindrical girders (III, FIG. 1), becomes extremely thin, preserving the width of the front part of the cranial support and increases in width entering the dorsal part of the parietal bones, so that it usually comprises a small portion of the lambdoid suture (II.1, FIG. 2). This way, excellent retention of our device in its place during activation is accomplished.

For more pressure comfort, a thin strip of soft medical silicone inlay is used (11.5, FIG. 1-4) on the inner side of the cranial support and of a such width, which corresponds to the width of the carbon fiber plate and of increased width in the dorsal occipital bone region (11.5, FIG. 2).

Afterwards, the horizontal girder, (IV, FIG. 1-5) is adjusted in the sagittal plane, which has an effect on the magnitude of the attached elastic traction force. Using the markings at its two ends (IV.5, FIG. 1, 5), ergonomic and absolute symmetrical adjustment on the right and left side of the patient's head is achieved. Its adjustment in height, which influences the angle of maxillary traction, is easily, precisely and absolutely symmetrically achieved on the right and left side of the patient's head, because of the semi-cylindrical slots (III.1, FIG. 1,2) in a fastener form, of the perpendicular cylindrical girders (III, FIG. 1,2). Its final immobilization within the perpendicular girders is achieved by means of small fixing screws (III.2, FIG. 1).

Finally, elastic traction bands (V, FIG. 1, 3-5) are applied to the maxilla, aiming its protraction. Initially, the elastic tractions are attached intraorally on the lateral hooks of the intraoral device and afterwards extraorally on the two mounting screws (IV.4, FIG. 1, 3-5) tightened in the metallic bar (IV.2, FIG. 1,4,5), which is integrated in the semi-cylindrical part of the horizontal carbon fiber girder between its bent points (IV, FIG. 1-5). The extraoral attachment of the elastic tractions is achieved ergonomically and absolutely symmetrically on the right and left side of the patient's head, also with respect to the patient's rima oris width, avoiding a synchilia's trauma, thanks to the many attachment positions in the whole width of the mouth slit.

In sum, the extraoral orthopaedic device in accordance with the disclosure is configured to help in the therapeutic modification of the maxillary and mandibular growth, in skeletal Class III and Class II patients, when it is used in cooperative growing patients.

Its main advantages are:

1. There is no adverse effect on the temporomandibular joint due to injury during its use, because of its skeletal anchorage only to the neurocranium during maxillary protraction and without any pressure on the mandible.

2. Ease of use, not only in the therapy of skeletal Class III patients, but for the aetiologic therapy of skeletal Class II patients, even in the cases of Angle Class I or Class II malocclusions with retrognathic maxilla and mandible within the face, which till now is not the case by using extraoral or intraoral devices.

3. Optimum symmetry in device settings, when the device is used on the patient's skull in a versatile and safe manner. The applying elastic tractions can be attached in the direction desired by the orthodontist both vertically and transversely, due to the ergonomic and practical design.

Lastly, parts of this device could be manufactured with other materials beyond those mentioned. Indicatively, the carbon fiber material, which is extremely lightweight and durable against breaking of the plate and girders, could be replaced by hard plastic compounds. The carbon fiber of the girders could be also replaced by metal. The dorsal part of the circular cranial support could be replaced by a soft self-fastening tape hook fastener (Velcro), aiming to remove the clamping mechanism from the front of the circular cranial support. The horizontal girder could have integrated protruding hooks in its whole frontal region for the easy attachment of the traction elastics, eliminating the integrated metallic bar, which was mentioned in the main design variant of our device.

In one general aspect, and as shown in the figures, the present disclosure describes an extraoral orthopaedic device for direct protraction of a maxilla and indirect protraction of a mandible of a patient. The device includes a circular cranial support configured for placement around a periphery of a neurocranium of the patient adjacent the forehead, two perpendicular girders slidably coupled to the circular cranial support and extending in a downward direction on either side of a face of the patient, each of the two perpendicular girders having a plurality of openings extending diametrically therethrough and disposed distally relative to the circular cranial support, an intraoral mechanism disposed in the mouth of the patient, and a horizontal girder coupled through elastic tractions with the intraoral mechanism and slidably supported within a respective one of the plurality of openings in each of the two perpendicular girders.

In one embodiment, the circular cranial support includes a clamping mechanism that abuts the neurocranium adjacent to the frontal bone area and its lateral parts. The circular cranial support further includes an adjustment mechanism for securing to the neurocranium, and support structures configured to symmetrically align the two perpendicular girders to the face of the patient. The horizontal girder is adjustably mountable to the two perpendicular girders to adjustably control an intensity of elastic tractions to the intraoral mechanism.

In one embodiment shown in the figures, the circular cranial support includes slots into which each of the two perpendicular girders and securable. Each of said slots is associated with a corresponding fixing screw configured to immobilize the respective one of the two perpendicular girders therein. The plurality of openings may form a slot for securing the horizontal girder to each of the two perpendicular girders. Each slot may further include a fixing screw.

In one embodiment, markings are disposed at the top end of each of the two perpendicular girders, the markings configured to symmetrically place the two perpendicular girders on the patient.

In one embodiment, each of the two perpendicular girders has a cylindrical cross section. Further, the horizontal girder may be generally U shaped and includes a metallic bar and two bent portions, the bent portions thereof being connectable to the two perpendicular girders.

The horizontal girder may have has a semi-circular cross section along the metallic bar and a circular cross section along the two bent portions, and may further include markings configured to symmetrically attach the horizontal girder to the two perpendicular girders symmetrically relative to the patient.

In one embodiment, a soft lining may be disposed along an inner surface of the circular cranial support that abuts the patient during use, and the soft lining may be made from medical silicone.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An extraoral orthopedic device for direct protraction of the maxilla and indirect protraction of the mandible of a patient, comprising:
a circular cranial support configured for placement around the periphery of the neurocranium of the patient encircling the forehead,
two perpendicular girders adjustably coupled to the circular cranial support and extending in a downward direction on either side of the face of the patient, each of the two perpendicular girders having a plurality of openings extending diametrically therethrough and disposed distally relative to the circular cranial support;
an intraoral mechanism disposed in the mouth of the patient; and
a horizontal girder coupled through elastic tractions with the intraoral mechanism and adjustably supported within the respective slot of the plurality of openings in each of the two perpendicular girders;
wherein the circular cranial support includes a clamping mechanism that abuts the neurocranium adjacent to the frontal bone area and its lateral parts; first means of adjustment and support top ensure the easy and symmetric placement of the two said perpendicular girders individualized to each patient.
wherein the horizontal girder is adjustably mountable to the two perpendicular girders to optimize and adjustably control the direction and intensity of elastic tractions to the intraoral mechanism, individualized to each patient.

2. The extraoral orthopedic device according to claim 1, wherein the circular cranial support includes slots into which each of the two perpendicular girders can be secured individualized to each patient.

3. The extraoral orthopedic device according to claim 2, wherein each of said slots is associated with a corresponding fixing screw configured to immobilize the respective one of the two perpendicular girders therein.

4. The extraoral orthopedic device according to claim 1, the plurality of openings provides a slot to secure the horizontal girder to each of the two perpendicular girders, aimed at the easy, symmetric and individualized adjustment to each patient.

5. The extraoral orthopedic device according to claim 4, wherein each slot further includes a fixing screw, aimed at immobilizing the horizontal girder after its adjustment to its final position.

6. The extraoral orthopedic device according to claim 1, further comprising two perpendicular girders with markings disposed at the top end of each, the markings configured to symmetrically place the two perpendicular girders on the patient.

7. The extraoral orthopedic device according to claim 1, wherein each of the two perpendicular girders has a cylindrical cross section.

8. The extraoral orthopedic device according to claim 1, wherein the horizontal girder is generally U shaped and includes a metallic bar and two bent portions. The metallic bar includes sockets in the form of internal threads, which accommodate the mounting screws, where the elastic tractions are attached extraorally.

9. The extraoral orthopedic device according to claim 8, wherein the horizontal girder has a semi-circular cross section along the metallic bar and a circular cross section along the two bent portions.

10. The extraoral orthopedic device according to claim 8, wherein the horizontal girder further includes markings configured to symmetrically attach the horizontal girder to the two perpendicular girders symmetrically relative to the patient.

11. The extraoral orthopedic device according to claim 1, wherein the outer hard portion of said circular cranial support, said perpendicular girders and said horizontal girder, apart from its incorporating metallic part, should conform to a construction material that has excellent mechanical properties and is very light in weight, preferably carbon fiber.

12. The extraoral orthopedic device according to claim 1, further comprising a soft lining disposed along an inner surface of the circular cranial support that abuts the patient during use, wherein the soft lining is made from medical silicone.
